(19) United States
(12) Patent Application Publication
(10) Pub. No.: US 2020/0306532 A1
(43) Pub. Date: Oct. 1, 2020

(54) SYSTEMS AND METHODS FOR THE MODULATION OF GUT MOTILITY

(71) Applicant: Duke University, Durham, NC (US)

(72) Inventors: Warren M. Grill, Durham, NC (US); Bradley Barth, Durham, NC (US); Craig Henriquez, Durham, NC (US); Xiling Shen, Durham, NC (US)

(21) Appl. No.: 16/624,673

(22) PCT Filed: Jun. 19, 2018
(86) PCT No.: PCT/US2018/038301

§ 371 Date: Dec. 19, 2019

Related U.S. Application Data
(60) Provisional application No. 62/521,658, filed on Jun. 19, 2017.

(51) Int. Cl. A61N 1/36; A61N 1/08

(52) U.S. Cl. CPC A61N 1/36007; A61N 1/3606; A61N 1/08

(57) ABSTRACT

The present disclosure relates to systems and methods for modulating gut motility. In particular, the present disclosure provides systems and methods for entraining interstitial cells of Cajal (ICCs) using patterns of electrical stimulation to increase gut motility. The systems and methods of ICC entrainment disclosed herein facilitate the treatment of various functional gastrointestinal disorders characterized by symptoms of dysmotility.

### RELATED APPLICATIONS

This application is a national phase application under 35 U.S.C. § 371 of PCT International Application No. PCT/US2018/038301, filed Jun. 19, 2018, which claims priority to and the benefit of U.S. Provisional Patent Application No. 62/521,658 filed Jun. 19, 2017, which is incorporated herein by reference in its entirety for all purposes.

### GOVERNMENT FUNDING

This invention was made with Government support under Federal Grant Nos. OT2-OD023849 and N66001-15-2-4059 awarded by the NIH and DARPA, respectively. The Federal Government has certain rights to the invention.

### FIELD

The present disclosure provides systems and methods relating to the modulation of gut motility. In particular, the present disclosure provides systems and methods for entraining interstitial cells of Cajal (ICCs) using patterns of electrical stimulation to increase gut motility. The systems and methods of ICC entrainment disclosed herein facilitate the treatment of various functional gastrointestinal disorders characterized by symptoms of dysmotility.

### BACKGROUND

Gut motility is required for digestion, homeostasis, and host-microbiome interactions, and it is regulated by the enteric nervous system (ENS). Pathology in the gut, especially in the ENS, can lead to functional gastrointestinal (GI) disorders characterized by symptoms of dysmotility that affect more than one fourth of the world's population. Implantable devices that electrically modulate neural function have demonstrated success including the cochlear implant, deep brain stimulation and spinal cord stimulation. However, electrical stimulation of the GI tract to treat gut dysmotility has seen limited success.

Although sacral nerve stimulation (SNS) has been used as a potential treatment for some GI disorders, such as constipation and irritable bowel syndrome, its effectiveness is limited by a lack of understanding of the effects of electrical stimulation on gut motility and ENS circuitry. Further, it is difficult to record and interpret the effects that stimulation has on the ENS in vivo. Electrical recordings from the ENS are challenging, at least in part due to noise contamination from smooth muscle contractions.

### SUMMARY

Embodiments of the present disclosure include a method of modulating gut motility in a subject. In accordance with these embodiments, the method includes applying a pattern of electrical stimulation to a portion of the subject's gastrointestinal tract, wherein the pattern of electrical stimulation entrains interstitial cells of Cajal (ICCs) in the portion of the subject's gastrointestinal tract and stimulates gut motility.

In some embodiments, portion of the subject's gastrointestinal tract includes the distal colon, rectum, or small intestine. In some embodiments, the pattern of electrical stimulation includes sinusoidal currents. In some embodiments, the sinusoidal currents are applied at from about 0.1 Hz to about 5.0 Hz. In some embodiments, the sinusoidal currents are applied at from about 0.1 Hz to about 1.0 Hz. In some embodiments, the sinusoidal currents are applied at about 0.5 Hz. In some embodiments, the sinusoidal currents are applied at from about 0.1 mA to about 25.0 mA. In some embodiments, the sinusoidal currents are applied at from about 1.0 mA to about 20.0 mA.

In other embodiments, the pattern of electrical stimulation includes pulse stimulations. In some embodiments, the pulse stimulations are applied at from about 0.1 Hz to about 20.0 Hz. In some embodiments, the pulse stimulations are applied at from about 0.5 Hz to about 15.0 Hz. In some embodiments, the pulse stimulations are applied at about 14.0 Hz. In some embodiments, the pulse stimulations are from about 100 μs to about 500 μs in duration. In some embodiments, the pulse stimulations are from about 100 μs to about 250 μs in duration. In some embodiments, the pulse stimulations are about 200 μs in duration. In some embodiments, the pulse stimulations are applied at from about 0.1 mA to about 25.0 mA. In some embodiments, the pulse stimulations are applied at from about 1.0 mA to about 20.0 mA.

In other embodiments, the method further includes treating one or more symptoms of a functional gastrointestinal and/or motility disorder in the subject. In some embodiments, the functional gastrointestinal and/or motility disorder is selected from the group consisting of: an esophageal disorder, a gastroduodenal disorder, a bowel disorder, centrally mediated disorders of gastrointestinal pain, gallbladder and sphincter of Oddi disorders, anorectal disorders, intestinal pseudo-obstructions, and childhood functional GI disorders. In some embodiments, the functional gastrointestinal and/or motility disorder includes chronic idiopathic constipation. In some embodiments, the one or more symptoms include gut dysmotility, and the application of the pattern of electrical stimulation to the portion of the subject's gastrointestinal tract treats the dysmotility. In some embodiments, the subject is a human.

Embodiments of the present disclosure include a method of treating a functional gastrointestinal disorder in a subject. In accordance with these embodiments, the method includes applying a pattern of electrical stimulation to a portion of the subject's gastrointestinal tract, wherein the pattern of electrical stimulation entrains interstitial cells of Cajal (ICCs) in the portion of the subject's gastrointestinal tract, and treating one or more symptoms of a functional gastrointestinal and/or motility disorder in the subject.

In some embodiments, the pattern of electrical stimulation includes sinusoidal currents. In some embodiments, the sinusoidal currents are applied at from about 0.1 Hz to about 5.0 Hz. In some embodiments, the sinusoidal currents are applied at about 0.5 Hz. In some embodiments, the sinusoidal currents are applied at from about 0.1 mA to about 25.0 mA.

In other embodiments, the pattern of electrical stimulation includes pulse stimulations. In some embodiments, the pulse stimulations are applied at from about 0.5 Hz to about 15.0 Hz. In some embodiments, the pulse stimulations are applied at about 14.0 Hz. In some embodiments, the pulse stimulations are from about 100 μs to about 250 μs in duration. In some embodiments, the pulse stimulations are about 200 μs in duration. In some embodiments, the pulse stimulations are applied at from about 0.1 mA to about 25.0 mA.

In other embodiments, the functional gastrointestinal and/or motility disorder is selected from the group consisting of: an esophageal disorder, a gastroduodenal disorder, a bowel disorder, centrally mediated disorders of gastrointestinal pain, gallbladder and sphincter of Oddi disorders, anorectal disorders, intestinal pseudo-obstructions, and childhood functional GI disorders. In some embodiments, the functional gastrointestinal and/or motility disorder includes chronic idiopathic constipation. In some embodiments, the one or more symptoms include dysmotility and the pattern of electrical stimulation includes sinusoidal currents, and wherein the application of the electrical stimulation to the portion of the subject's gastrointestinal tract stimulates gut motility. In some embodiments, the subject is a human.

Embodiments of the present disclosure include a method of operating an implantable neuromodulation device to treat a functional gastrointestinal and/or motility disorder in a subject. In accordance with these embodiments, the method includes configuring the neuromodulation device to apply a pattern of electrical stimulation to a portion of a subject's gastrointestinal tract, wherein the pattern of electrical stimulation entrains interstitial cells of Cajal (ICCs) in the portion of the subject's gastrointestinal tract, and treating one or more symptoms of the functional gastrointestinal and/or motility disorder in the subject.

In some embodiments, the pattern of electrical stimulation includes sinusoidal currents. In some embodiments, the sinusoidal currents are applied at from about 0.1 Hz to about 5.0 Hz. In some embodiments, the sinusoidal currents are applied at about 0.5 Hz. In some embodiments, the sinusoidal currents are applied at from about 0.1 mA to about 25.0 mA.

In other embodiments, the pattern of electrical stimulation includes pulse stimulations. In some embodiments, the pulse stimulations are applied at from about 0.5 Hz to about 15.0 Hz. In some embodiments, the pulse stimulations are applied at about 14.0 Hz. In some embodiments, the pulse stimulations are from about 100 μs to about 250 μs in duration. In some embodiments, the pulse stimulations are about 200 μs in duration. In some embodiments, the pulse stimulations are applied at from about 0.1 mA to about 25.0 mA.

In other embodiments, the functional gastrointestinal and/or motility disorder is selected from the group consisting of: an esophageal disorder, a gastroduodenal disorder, a bowel disorder, centrally mediated disorders of gastrointestinal pain, gallbladder and sphincter of Oddi disorders, anorectal disorders, intestinal pseudo-obstructions, and childhood functional GI disorders. In some embodiments, the functional gastrointestinal and/or motility disorder includes chronic idiopathic constipation. In some embodiments, the one or more symptoms include dysmotility and the pattern of electrical stimulation includes sinusoidal currents, and wherein the application of the electrical stimulation to the portion of the subject's gastrointestinal tract stimulates gut motility. In some embodiments, the subject is a human.

### BRIEF DESCRIPTION OF THE DRAWINGS

FIGS. 1A-1C include representative schematics of the computational model and corresponding results of gut motility stimulation of peristalsis. FIG. 1A: Illustration of pellet moving through a section of the GI tract. FIG. 1B: Network of model enteric neurons, smooth muscle, and ICCs. SN: sensory neuron, AI: ascending interneuron, DI: descending interneuron, IN: inhibitory motor neuron, EN: excitatory motor neuron, CM: circular muscle fiber. FIG. 1C: Pellet position over time, along with raster plots of sensory neuron action potentials and circular muscle fiber contractions, as functions of position.

FIGS. 2A-2D include representative results of the effects of electrical stimulation on transit time. FIG. 2A: Pellet position, circular muscle activity, and sensory neuron activity over time during 14 Hz, 200 μs pulse stimulation at 1 mA. FIG. 2B: Sensory neuron action potentials during pulse stimulation in FIG. 2A. FIG. 2C: Circular muscle subthreshold oscillations during pulse stimulation in FIG. 2A. FIG. 2D: Pellet position, circular muscle activity, and sensory neuron activity over time during 14 Hz sine stimulation at 1 mA.

FIGS. 3A-3E include representative results characterizing sine wave stimulation over a range of frequencies. Pellet position, circular muscle activity, and sensory neuron activity during 0.5 Hz (FIG. 3A), 5 Hz (FIG. 3B), and 50 Hz (FIG. 3C) sine wave stimulation at 1 mA. FIG. 3D: Motility speed as percent control for each of the sine wave stimulation frequencies. FIG. 3E: Comparing motility speed between the optimal sine wave stimulation (0.5 Hz) and 14 Hz and 0.5 Hz pulse stimulation.

FIGS. 4A-4F include representative results characterizing the role of ICCs in electrical stimulation of gut motility. FIG. 4A: Threshold current used to entrain ICC pacemaker frequency to match sine stimulation frequency. Membrane potential of the ICC at position L/4 in the model of motility during no stimulation (FIG. 4B), 0.5 Hz sine wave stimulation (FIG. 4C), and 0.5 Hz (FIG. 4D), with 200 μs pulse stimulation at 1 mA. Pellet position, circular muscle activity, and sensory neuron activity during 0.5 Hz, 1 mA sine wave stimulation with electrical stimulation influencing enteric neurons and smooth muscle, but not ICCs (FIG. 4E), and influencing only ICCs, but not enteric neurons and smooth muscle (FIG. 4F).

FIGS. 5A-5D include representative results of the effects of stimulation in intermediate and advanced models of motility. Motility speed (% control) is compared in the intermediate expanded model between 0.5 Hz, 5 Hz, and 50 Hz sine wave stimulation (FIG. 5A) and 14 Hz and 0.5 Hz pulse stimulation at 1 mA (FIG. 5B). In the advanced model, motility speed (% control) is compared between 0.5 Hz, 5 Hz, and 50 Hz sine wave stimulation (FIG. 5C) and 14 Hz and 0.5 Hz pulse stimulation at 1 mA (FIG. 5D). N=9 for all groups; error bars show standard error.

FIGS. 6A-6D include representative results of the effects of electrical stimulation on colonic transit time in awake rats. FIG. 6A: Individual trials for colon transit time during sine wave stimulation. FIG. 6B: Summary statistics for mean motility speed as percent control during sine wave stimulation. FIG. 6C: Individual trials for colon transit time during 0.5 Hz sine wave stimulation compared to pulse stimulation. FIG. 6D: Summary statistics comparing mean motility speed as percent control between 0.5 Hz sine wave and pulse stimulation. N=7 for all groups; error bars show standard error. Star (★) denotes significant difference between control (unstimulated) and experimental (stimulated) groups, as determined by two-tailed, paired t-test. Dagger (†) denotes significantly different from all other groups, as determined by ANOVA and Tukey HSD post hoc testing.

FIGS. 7A-7D include representative results of excitation properties of biophysically-based model cells. FIG. 7A: Action potential in Connor-Stevens model neuron, stimulated with 7.5 nA intracellular current injection. FIG. 7B: Smooth muscle fiber contraction, stimulated with 25 nA intracellular current injection. FIG. 7C: Smooth muscle fiber tension when membrane potential is greater than threshold (solid) and less than threshold (dashed). FIG. 7D: ICC membrane potential without stimulation.

FIGS. 8A-8E include representative results of properties of connections between model cells. FIG. 8A: Action potential in presynaptic neuron (blue) triggers an action potential in the postsynaptic neuron (black). FIG. 8B: ICC pacemaker activity (blue) causes depolarizations in circular muscle (red) and longitudinal muscle (green) through coupling via gap junctions. FIG. 8C: Excitatory motor neuron (blue) cause excitatory junction potentials (EJP) that do not evoke a contraction in smooth muscle fiber (red) without ICC coupling. FIG. 8D: EJPs cause contraction in smooth muscle when the fiber is coupled to ICCs with gap junctions. FIG. 8E: Smooth muscle contraction caused by EJPs while coupled to ICCs is blocked by an inhibitory junction potential (IJP) from inhibitory motor neuron (black).

FIGS. 9A-9D include a representative schematic of the virtual pellet model and its corresponding functional characterization. FIG. 9A: Schematic of 2D pellet. FIG. 9B: Contribution weight of each muscle fiber to tension at the pellet endcaps, determining angle β, as a function of distance between fiber and endcap. FIG. 9C: Distension of intestinal wall, contributing to stretch current in sensory neurons, as a function of distance between sensory neuron and the center of the pellet. FIG. 9D: Sensory neuron membrane action potentials when distension (D) is equal to 1 (located at the pellet).

FIG. 10 includes representative results of activating function as a function of distance from the modeled point-source electrode. Vertical line indicates y-distance between electrode and cells in the motility model, 750 μm.

FIGS. 11A-11C include representative results of current thresholds and firing rates for stimulation of single, isolated model cells. Threshold current was determined for smooth muscle fibers (circles) and enteric neurons (squares) for pulse stimulation (FIG. 11A), and sine wave stimulation (FIG. 11B). Mean firing rate for neurons and smooth muscle plotted against frequency for 200 μs pulse and sine wave stimulation at 1 mA (FIG. 11C).

FIG. 12 includes a representative schematic of the surgical procedure used to implant electrodes in rat models.

FIGS. 13A-13E include representative results using the stimulated motility model with scaled current amplitude. Pellet position, circular muscle activity, and sensory neuron activity during 50 Hz sine wave stimulation at 3 mA (FIG. 13A), 14 Hz pulse stimulation at 16.3 mA (FIG. 13B), 0.5 Hz pulse stimulation at 1 mA (FIG. 13C), 0.5 Hz pulse stimulation at 16.3 mA (FIG. 13D), and 0.1 Hz sine wave stimulation at 1 mA (FIG. 13E).

FIGS. 14A-14G include representative results of unstimulated pellet propagation with additional mechanisms. Pellet position, circular muscle activity, and sensory neuron activity in unstimulated models with added mechanisms: increased junction potential conductance (FIG. 14A), extrinsic feedback loop and ascending inhibition (FIG. 14B), descending excitation (FIG. 14C), fiber conduction delays (FIG. 14D), sensory afterhyperpolarization current (FIG. 14E), graded sensory stretch response (FIG. 14F), and stochastic, spontaneous excitatory and inhibitory junction potentials (FIG. 14G).

FIGS. 15A-15C include representative results of afterhyperpolarization and graded sensory response mechanisms in sensory neurons. Membrane potential (mV) traces of sensory neurons responding to low (FIG. 15A) and high (FIG. 15B) degrees of stretch applied for 4 seconds, initiated at 1 second. Pellet velocity (% change) for increasing pellet sizes in the expanded model with AH mechanisms and graded stretch response (FIG. 15C).

### DETAILED DESCRIPTION

The present disclosure provides systems and methods relating to the modulation of gut motility. In particular, the present disclosure provides systems and methods for entraining interstitial cells of Cajal (ICCs) using patterns of electrical stimulation to increase gut motility. The systems and methods of ICC entrainment disclosed herein facilitate the treatment of various functional gastrointestinal disorders characterized by symptoms of dysmotility.

Embodiments of the present disclosure provide, in part, a computational model that integrates electrical slow wave propagation with a network model of the ENS. Unlike models currently being used, the model provided herein integrates enteric neurons, interstitial cells of Cajal (ICCs), and smooth muscle in an interconnected network. The model was designed to recreate gut motility (e.g., peristalsis) through the ascending excitatory and descending inhibitory pathways, where enteric neurons integrate sensory feedback to control smooth muscle contraction. Gut motility was simulated though the incorporation a virtual pellet that interacted with the network by stimulating sensory neurons and responding to smooth muscle contractions.

Embodiments of the integrated neuromechanical model described herein accurately simulated the effects of electrical stimulation on gut motility, which demonstrated that certain patterns of electrical stimulation (e.g., sinusoidal currents, pulse stimulation) can be used to entrain ICCs. Effective ICC entrainment can, in turn, be used to treat gut dysmotility. In some embodiments, sinusoidal currents at 0.5 Hz were more effective at accelerating gut motility than commonly used higher frequency current pulses; however, both electrical stimulation comprising either sine currents or pulse stimulations were effective at increasing gut motility to ICC entrainment as compared to no stimulation. The computation models described herein were substantiated by in vivo experiments measuring colon transit time in awake rats during electrical stimulation.

Embodiments of the present disclosure involve the development of an integrated neuromechanical model comprising simplified biophysical representations of enteric neurons, ICCs, smooth muscle, and mechanical interaction with a virtual pellet. In accordance with these embodiments, the modeling described herein captures electrical slow wave propagation, sensory feedback, smooth muscle contraction, and motility. In one embodiment, this model predicted that low-frequency (e.g., 0.5 Hz) sine wave stimulation would be effective at enhancing gut motility compared to higher-frequency sine waves and conventional biphasic pulse stimulation, despite pulses being more effective at stimulating neurons. This prediction was validated by in vivo measurements of colon transit time in awake rats. The stimulation strategy to enhance gut motility identified with the model is novel as compared to currently used neuromodulation parameters that are thought to activate neurons most effectively.

In some embodiments, the model demonstrated that low-frequency sine wave stimulation was more effective at modulating (e.g., entraining) the natural pacemaker frequency of the ICCs to a higher frequency, hence accelerating gut motility. Threshold currents for entraining ICCs were lower for sine wave at frequencies closer to the 0.1 Hz natural ICC pacemaker frequency, and ICCs responded to sine wave stimulation over greater distances than to equal amplitude pulse stimulation. This mechanism offers an explanation as to why sine wave stimulation entrained pacemaker activity more effectively than pulse stimulation, even though both patterns of stimulation effectively entrained ICCs as compared to no stimulation.

The limitation of current experimental methods supports the importance of using computational modeling as a tool to gain understanding, and this is a strength of this modeling approach described herein. The model provided insights into potential mechanisms behind functional electrical stimulation that would be challenging to deduce from experimental measurements alone, and predictions from the model were qualitatively validated by in vivo experimental data, and the predictions are biophysically relevant for therapeutic development.

The integrated neuromechanical model of the present disclosure offers insight into the mode of action for increasing gut motility by electrical stimulation. The model demonstrated that by affecting the frequency of pacemaker ICCs, low-frequency sine wave stimulation was more effective at increasing gut motility than conventional current pulses, and this prediction was verified in awake animal studies. The computational model provides a platform to explore a wide array of stimulation patterns and parameters more efficiently than empirical approaches, and it may lead to refinement of current clinical approaches to neuromodulation in the gastrointestinal tract and enteric nervous system.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

“Entrain” or “entrainment” as used herein refers to a process of altering a subject's biological rhythm to assume a different cycle or frequency. “Entrain” or “entrainment” as used herein also refers to altering a biological rhythm that is symptomatic of disease, such as, but not limited to, entraining one or more pacemaker cells (e.g., ICCs) to match the frequency of applied patterns of electrical stimulation to treat one or more symptoms of the disease.

“Gastrointestinal tract motility” or “gut motility” as used herein refers to the movements of the digestive system and the transit of the contents within it. Accordingly, when nerves and/or muscles in any portion of the digestive tract do not function normally (e.g., abnormal strength and/or coordination), a subject can develop one or more symptoms related to guy dysmotility.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Methods of Modulating of Gut Motility

Embodiments of the present disclosure include methods of modulating gut motility in a subject. In accordance with these embodiments, the method includes applying a pattern of electrical stimulation to a portion of the subject's gastrointestinal tract to modulate gut motility and/or treat gut dysmotility. In some embodiments, the application of certain patterns of electrical stimulation entrains interstitial cells of Cajal (ICCs) in the portion of the subject's gastrointestinal tract and modulates gut motility and/or treats gut dysmotility. ICCs include a type of interstitial cell found in the gastrointestinal tract, with different types with different functions. For example, myenteric Interstitial cells of Cajal (ICC-MY) serve as a pacemaker which creates the bioelectrical slow wave potential that leads to contraction of the smooth muscle. The frequency of ICC pacemaker activity differs in different regions of the GI tract: about 3 per minute in the stomach; about 11-12 per minute in the duodenum; about 8-9 per minute in the ileum; and about 3-4 per minute in the colon. Intramuscular Interstitial cells of Cajal (ICC-IM) are involved in the stimulation of smooth muscle cells, and neurotransmitters can act through them. Many types of smooth muscle tissues have been shown to contain ICC, but with certain exceptions, the function of ICCs cells has not been fully elucidated. Accordingly, embodiments of the present disclosure include the application of certain patterns of electrical stimulation to entrain ICCs in a portion of a subject's gastrointestinal tract, including, for example, in the colon, the distal colon, the rectum, the stomach, the ileum, the duodenum, and/or the small intestine.

In some embodiments, the pattern of electrical stimulation includes sinusoidal currents. In some embodiments, the application of electrical stimulation comprising sinusoidal currents entrains ICCs and modulates gut motility and/or treats gut dysmotility. Sinusoidal currents can be applied at various frequencies and amplitudes. For example, in some embodiments, sinusoidal currents are applied at from about 0.1 Hz to about 5.0 Hz; from about 0.1 Hz to about 4.0 Hz; from about 0.1 Hz to about 3.0 Hz; from about 0.1 Hz to about 2.0 Hz; from about 0.1 Hz to about 1.0 Hz; or from about 0.1 Hz to about 0.5 Hz. In some cases, sinusoidal currents are applied at about 0.5 Hz.

In accordance with these embodiments, sinusoidal currents can be applied at various amplitudes, including but not limited to from about 0.1 mA to about 25.0 mA; from about 0.1 mA to about 20.0 mA; from about 0.1 mA to about 15.0 mA; from about 0.1 mA to about 10.0 mA; from about 0.1 mA to about 5.0 mA; from about 0.1 mA to about 4.0 mA; from about 0.1 mA to about 3.0 mA; from about 0.1 mA to about 2.0 mA; from about 0.1 mA to about 1.0 mA. In some cases, sinusoidal currents are applied at about 1.0 mA.

In some embodiments, the pattern of electrical stimulation includes pulse stimulation. In some embodiments, the application of electrical stimulation comprising pulse stimulation entrains ICCs and modulates gut motility and/or treats gut dysmotility. Pulse stimulations can be applied at various frequencies and amplitudes. For example, in some embodiments, pulse stimulations are applied at from about 0.1 Hz to about 20.0 Hz; from about 0.1 Hz to about 15.0 Hz; from about 0.1 Hz to about 10.0 Hz; from about 0.1 Hz to about 5.0 Hz; from about 0.1 Hz to about 1.0 Hz; from about 1.0 Hz to about 20.0 Hz; from about 5.0 Hz to about 20.0 Hz; from about 5.0 Hz to about 15.0 Hz; or from about 0.5 Hz to about 15.0 Hz. In some cases, pulse stimulations are applied at about 14.0 Hz.

In accordance with these embodiments, pulse stimulation can be applied at various amplitudes, including but not limited to from about 0.1 mA to about 25.0 mA; from about 0.1 mA to about 20.0 mA; from about 0.1 mA to about 15.0 mA; from about 0.1 mA to about 10.0 mA; from about 0.1 mA to about 5.0 mA; from about 0.1 mA to about 4.0 mA; from about 0.1 mA to about 3.0 mA; from about 0.1 mA to about 2.0 mA; from about 0.1 mA to about 1.0 mA. In some cases, pulse stimulations are applied at about 1.0 mA.

In accordance with these embodiments, pulse stimulation can be applied for various durations or lengths of time, including but not limited to, for about 100 μs to about 500 μs; for about 100 μs to about 450 μs; for about 100 μs to about 400 μs; for about 100 μs to about 350 μs; for about 100 μs to about 300 μs; for about 100 μs to about 250 μs; or for about 100 μs to about 200 μs. In some cases, pulse stimulations are applied for about 200 μs.

In some embodiments, methods of modulating gut motility and/or treating gut dysmotility in a subject include treating one or more symptoms of a functional gastrointestinal and/or motility disorder in the subject. In accordance with these embodiments, a functional gastrointestinal and/or motility disorder can include, but is not limited to, an esophageal disorder, a gastroduodenal disorder, a bowel disorder, centrally mediated disorders of gastrointestinal pain, gallbladder and sphincter of Oddi disorders, anorectal disorders, intestinal pseudo-obstructions, and childhood functional GI disorders. In some cases, the functional gastrointestinal and/or motility disorder comprises chronic idiopathic constipation.

In some embodiments, methods of modulating gut motility and/or treating gut dysmotility in a subject include treating one or more symptoms of a functional gastrointestinal and/or motility disorder. In some cases, the one or more symptoms of the functional gastrointestinal and/or motility disorder comprise gut dysmotility, and the application of a pattern of electrical stimulation to a portion of a subject's gastrointestinal tract treats the dysmotility, which can include the entrainment of ICCs. In some embodiments, the subject being treated is a human subject in need of treatment, such as a human patient suffering from a functional gastrointestinal and/or motility disorder that comprises chronic idiopathic constipation.

Embodiments of the present disclosure also include a method of treating a functional gastrointestinal disorder in a subject. In accordance with these embodiments, the method can include applying a pattern of electrical stimulation to a portion of the subject's gastrointestinal tract such that the pattern of electrical stimulation entrains interstitial cells of Cajal (ICCs) in the portion of the subject's gastrointestinal tract. Entraining ICCs by application of a pattern of electrical stimulation can treat one or more symptoms of a functional gastrointestinal and/or motility disorder in the subject.

Treating a functional gastrointestinal and/or motility disorder in the subject can include reversing, alleviating, or inhibiting the progress of the disorder, or one or more symptoms of such disorder. Depending on the condition of the subject, treatment also includes preventing the functional gastrointestinal and/or motility disorder in the subject, and includes preventing the onset of the disorder, or preventing the symptoms associated with the disorder. A treatment may be either performed in an acute or chronic way, as described herein, and may be administered by a physician as part of a treatment regimen and/or in conjunction with other treatments. Treating a functional gastrointestinal and/or motility disorder in a subject also includes reducing the severity of a disorder or symptoms associated with the disorder prior to being diagnosed or afflicted with the disorder. Such prevention or reduction of the severity of a functional gastrointestinal and/or motility disorder prior to diagnosis/affliction refers to administration of a treatment (e.g., electrical stimulation) to a subject that is not at the time of administration afflicted with the disorder.

### 3. Methods of Operating a Neuromodulation Device to Treat Functional Gastrointestinal and/or Motility Disorders

Embodiments of the present disclosure include a method of operating an implantable neuromodulation device to treat a functional gastrointestinal and/or motility disorder in a subject. In accordance with these embodiments, the method includes configuring a neuromodulation device to apply a pattern of electrical stimulation to a portion of a subject's gastrointestinal tract, such the pattern of electrical stimulation entrains interstitial cells of Cajal (ICCs) in the portion of the subject's gastrointestinal tract and treats one or more symptoms of the functional gastrointestinal disorder in the subject.

In some embodiments, operating an implantable neuromodulation device includes the application of a pattern of electrical stimulation that includes sinusoidal currents. In some embodiments, the application of electrical stimulation comprising sinusoidal currents using the neuromodulation device entrains ICCs and modulates gut motility and/or treats gut dysmotility. Sinusoidal currents can be applied at various frequencies and amplitudes. For example, in some embodiments, sinusoidal currents are applied at from about 0.1 Hz to about 5.0 Hz; from about 0.1 Hz to about 4.0 Hz; from about 0.1 Hz to about 3.0 Hz; from about 0.1 Hz to about 2.0 Hz; from about 0.1 Hz to about 1.0 Hz; or from about 0.1 Hz to about 0.5 Hz. In some cases, sinusoidal currents are applied at about 0.5 Hz.

In accordance with these embodiments, sinusoidal currents can be applied at various amplitudes, including but not limited to from about 0.1 mA to about 25.0 mA; from about 0.1 mA to about 20.0 mA; from about 0.1 mA to about 15.0 mA; from about 0.1 mA to about 10.0 mA; from about 0.1 mA to about 5.0 mA; from about 0.1 mA to about 4.0 mA; from about 0.1 mA to about 3.0 mA; from about 0.1 mA to about 2.0 mA; from about 0.1 mA to about 1.0 mA. In some cases, sinusoidal currents are applied at about 1.0 mA.

In some embodiments, the pattern of electrical stimulation includes pulse stimulation. In some embodiments, the application of electrical stimulation comprising pulse stimulation using the neuromodulation device entrains ICCs and modulates gut motility and/or treats gut dysmotility. Pulse stimulations can be applied at various frequencies and amplitudes. For example, in some embodiments, pulse stimulations are applied at from about 0.1 Hz to about 20.0 Hz; from about 0.1 Hz to about 15.0 Hz; from about 0.1 Hz to about 10.0 Hz; from about 0.1 Hz to about 5.0 Hz; from about 0.1 Hz to about 1.0 Hz; from about 1.0 Hz to about 20.0 Hz; from about 5.0 Hz to about 20.0 Hz; from about 5.0 Hz to about 15.0 Hz; or from about 0.5 Hz to about 15.0 Hz. In some cases, pulse stimulations are applied at about 14.0 Hz.

In accordance with these embodiments, pulse stimulation can be applied at various amplitudes, including but not limited to from about 0.1 mA to about 25.0 mA; from about 0.1 mA to about 20.0 mA; from about 0.1 mA to about 15.0 mA; from about 0.1 mA to about 10.0 mA; from about 0.1 mA to about 5.0 mA; from about 0.1 mA to about 4.0 mA; from about 0.1 mA to about 3.0 mA; from about 0.1 mA to about 2.0 mA; from about 0.1 mA to about 1.0 mA. In some cases, pulse stimulations are applied at about 1.0 mA.

In accordance with these embodiments, pulse stimulation can be applied for various durations or lengths of time, including but not limited to, for about 100 μs to about 500 μs; for about 100 μs to about 450 μs; for about 100 μs to about 400 μs; for about 100 μs to about 350 μs; for about 100 μs to about 300 μs; for about 100 μs to about 250 μs; or for about 100 μs to about 200 μs. In some cases, pulse stimulations are applied for about 200 μs.

In some embodiments, methods of modulating gut motility and/or treating gut dysmotility in a subject using the neuromodulation device include treating one or more symptoms of a functional gastrointestinal and/or motility disorder in the subject. In accordance with these embodiments, a functional gastrointestinal and/or motility disorder can include, but is not limited to, an esophageal disorder, a gastroduodenal disorder, a bowel disorder, centrally mediated disorders of gastrointestinal pain, gallbladder and sphincter of Oddi disorders, anorectal disorders, intestinal pseudo-obstructions, and childhood functional GI disorders. In some cases, the functional gastrointestinal and/or motility disorder comprises chronic idiopathic constipation.

In some embodiments, methods of modulating gut motility and/or treating gut dysmotility in a subject using the neuromodulation device include treating one or more symptoms of a functional gastrointestinal and/or motility disorder. In some cases, the one or more symptoms of the functional gastrointestinal and/or motility disorder comprise gut dysmotility, and the application of a pattern of electrical stimulation to a portion of a subject's gastrointestinal tract treats the dysmotility, which can include the entrainment of ICCs. In some embodiments, the subject being treated is a human subject in need of treatment, such as a human patient suffering from a functional gastrointestinal and/or motility disorder that comprises chronic idiopathic constipation.

In accordance with these embodiments, an implantable neuromodulation device to treat a functional gastrointestinal and/or motility disorder in a subject can include a neuromodulation system comprising one or more implantable electrodes and a signal generator device. In some embodiments, the system further comprises electrical terminals configured for being respectively coupled to a plurality of electrodes implanted within tissue (e.g., gastrointestinal tissue), analog output circuitry configured for delivering therapeutic electrical energy between the plurality of electrical terminals in accordance with a set of modulation parameters that includes a defined current value (e.g., a user-programmed value), and a voltage regulator configured for supplying an adjustable compliance voltage to the analog output circuitry. The neuromodulation device and/or system can further comprises control/processing circuitry configured for performing a compliance voltage calibration process at a compliance voltage adjustment interval by periodically computing an adjusted compliance voltage value as a function of a compliance voltage margin, directing the voltage regulator to adjust the compliance voltage to the adjusted compliance voltage value, and for adjusting at least one of the compliance voltage adjustment interval and the compliance voltage margin during the voltage compliance calibration process. The compliance voltage adjustments may be automatically performed as described above or manually performed in response to user input.

### 4. Materials and Methods

Several models of the enteric motor patterns of the ENS exist. Fundamental mechanisms for the ascending excitatory pathway were revealed in early simulations, and these were later expanded to include neural control of circular smooth muscle. Additional models were developed to study neural control of motility, including models for segmentation and migrating motor complexes. Separate models captured electrical slow waves that arise from the interaction between interstitial cells of Cajal (ICC) and smooth muscle fibers, but these models did not consider enteric neurons or neuromuscular junctions. Despite these efforts, there is no model that includes all the components necessary to capture the effect of electrical stimulation on gut motility, including conductance-based models of the electrical activity of enteric neurons and ICC-driven slow wave propagation through smooth muscle. Therefore, there is a need to create an accurate model of gut motility in the ENS to enable exploration of stimulation parameters and the mechanistic underpinnings of GI stimulation.

Computational Model. The model used herein included biophysically-based representations of individual cells, classified as enteric neurons, smooth muscle fibers, and ICCs. Neurons, muscle fibers, and ICCs were modeled as point cells in a 2D plane orthogonal to the circumference of a 10-cm length of the GI tract. The model was implemented by simulating the transmembrane potential of 196 individual point cells. The governing ODEs for the various cell types were solved using the exponential Euler method at a time step of 0.1 ms using the Brian 2 simulation environment in Python (see, e.g., Stimberg et al., 2013). The model assumed axial symmetry and collapsed the circumferential component of the GI tract onto a single point. The transmembrane potential (Vₘ) in each model cell was defined by a differential equation as a function of synaptic currents (I_{syn}), ionic membrane currents (Iᵢₒₙ), and membrane capacitance (Cₘ) (equation 1). In addition to the biophysical cells, the model also included representations of a pellet and a stimulating electrode.

$$\frac{d {V}_{m}}{d t} = \frac{{I}_{i o n} + {I}_{s y n}}{{C}_{m}} \qquad Equation 1$$

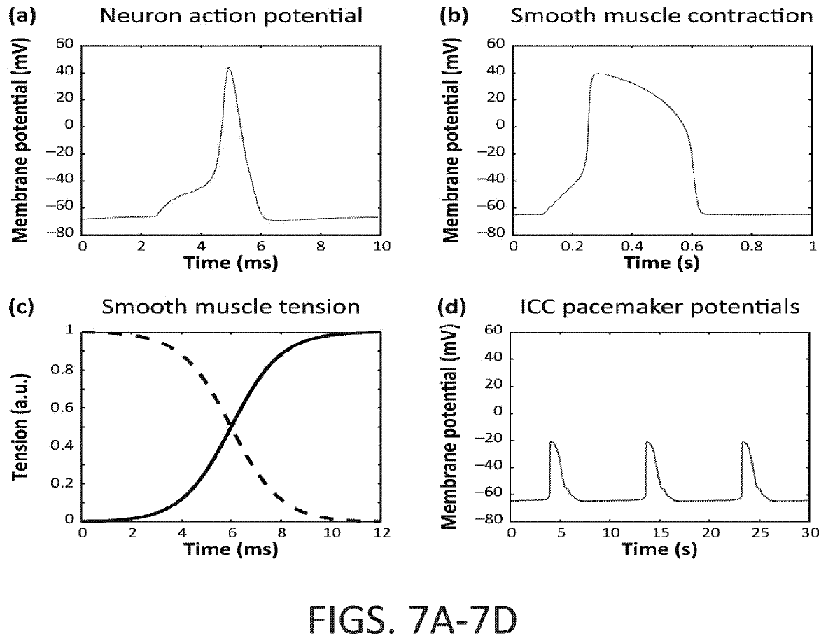

Enteric Neurons. Enteric neurons were modeled as Connor-Stevens neurons (see, e.g., Connor and Stevens, 1971). In addition to the leak channels (Iₗₑₐₖ) and voltage-gated sodium (I_{Na}) and potassium (I_{K}) channels of the Hodgkin-Huxley model, the Connor-Stevens model includes an additional transient potassium current (I_{KA}) (equation 2), which allowed for a broader range and tunability of firing rates (see, e.g., Drion et al., 2015). Enteric neurons received synaptic currents to simulate excitatory postsynaptic potentials (I_{EPSP}). The enteric neurons exhibited a firing threshold of approximately −35 mV (FIG. 7A). The sensory neurons in the ENS are characterized by an afterhyperpolarization (AH) that limits the maximum firing rate, which was not included in the Connor-Stevens model. The AH mechanism was later added to the Connor-Stevens model of sensory cells in the expanded model.

Iᵢₒₙ=Iₗₑₐₖ+I_{Na}+I_{K}+I_{KA} Equation 2

Smooth Muscle Fibers. Smooth muscle fibers were defined by a conductance model with ionic membrane currents and synaptic currents. Membrane currents include leak channels, potassium channels, and an L-type calcium channel (I_{CaL}) adapted from Tong et al. (2011) (equation 3(a)). The L-type calcium current cause long-duration depolarization in muscle fibers to model the depolarization-contraction relationship. Synaptic currents included excitatory junction potentials (I_{EJP}), inhibitory junction potentials (I_{IJP}), and gap junctions (I_{gap}) (equation 3(b)). Smooth muscle fibers exhibited contractions that lasted for a few hundred milliseconds and initiated contraction at approximately −36 mV (FIG. 7B).

Iᵢₒₙ=Iₗₑₐₖ+I_{K}+I_{CaL} Equation 3(a)

I_{syn}=I_{EJP}+I_{IJP}+I_{gap} Equation 3(b)

Smooth muscle contraction caused local tension which was defined for each muscle fiber as a continuous variable from [0, 1], with 1 indicating maximum tension. Previously, Ozaki et al. (1991) demonstrated the relationship between smooth muscle tension and membrane potential was a function of [Ca²⁺]. This mechanism was studied in silico by modeling [Ca²⁺] cellular-electrochemical coupling and sub-cellular mechanics, and this revealed that tension could be modeled as a normalized and sigmoidal function in time (see, e.g., Du et al., 2011). Therefore, tension was modeled as a sigmoid function in time, increasing or decreasing when membrane potential was above or below contraction threshold, respectively (FIG. 7C).

Interstitial Cells of Cajal (ICCs). ICCs were based on the model developed by Edwards and Hirst that included a leak current (Iₗₑₐₖ) and a pacemaker current (I_{pacemaker}) (equation 4). The Edwards and Hirst model for ICCs was chosen because it was developed from experimental analysis of pacemaker activity and slow wave propagation (see, e.g., Edwards and Hirst, 2006, Hirst et al., 2006). In this model, ICCs did not generate action potentials or contraction events, but due to the pacemaker current, ICCs exhibited regular depolarizations that occurred approximately every 10 s (FIG. 7D).

Iᵢₒₙ=Iₗₑₐₖ+I_{pacemaker} Equation 4

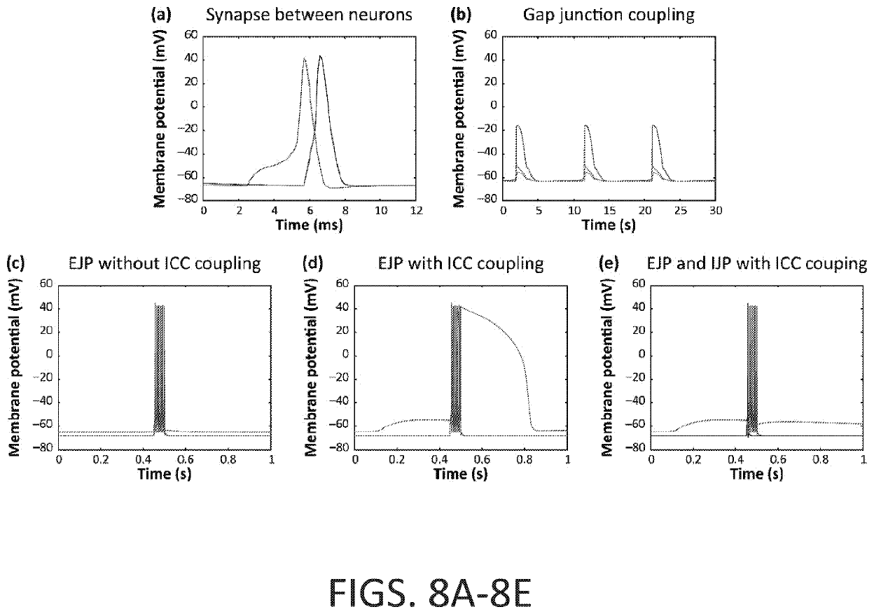

Connections. The model uses longitudinally positioned sensory and motor subnetworks including model cells connected by synapses, gap junctions, and neuromuscular junctions. Existing models have modeled the spatial dynamics of ENS and slow wave behavior by spacing subpopulations of cells longitudinally (see, e.g., Lin et al., 2006, Chambers et al., 2008, Du et al., 2011, Chambers et al., 2014). In the present disclosure, sensory and motor subnetworks were spatially distributed along the simulated GI tract to reflect GI anatomy and interneuron projection lengths in small rodents (see, e.g., Brookes et al., 1997, Permezel and Webling, 1971). Twelve independent sensory subnetworks were constructed to include enteric neurons connected by excitatory synapses. Excitatory synapses were modeled as conductance-based alpha synapses with reversal potentials depolarized to the resting potential (FIG. 8A). Sensory neurons were connected to local ascending and descending interneurons by excitatory synapses. Ascending interneurons innervated neighboring excitatory circular motor neurons orally, and descending interneurons innervated neighboring inhibitory circular motor neurons anally. Previous work has used these distinct neural populations and ascending excitatory and descending inhibitory pathways to model the neural mechanisms controlling motility (see, e.g., Bornstein et al., 1997, Bornstein et al., 2010, Chambers et al., 2011).

Smooth muscle fibers were connected to ICCs by gap junctions and were connected to motor neurons by neuromuscular junctions to form 40 identical motor subnetworks to reflect electrical coupling space constants in smooth muscle (see, e.g., van Helden and Imtiaz, 2003, Ward et al., 2003, Huizinga et al., 1988). Gap junctions connected ICCs to circular muscle fibers. The gap junctions were modeled as passive leak currents between two cells (FIG. 8B). The gap junctions connected cells at the same x-position; therefore, muscle fibers were only influenced by the pacemaker potential of the local ICCs, and not distal ICCs.

Neuromuscular junctions connected enteric motor neurons to smooth muscle fibers. Junction potentials were either excitatory (EJP) or inhibitory (IJP), with reversal potentials above or below the resting potential, respectively. EJPs were not strong enough to cause a contraction unless the motor neuron fired during the peak of pacemaker activity, but IJPs were strong enough to block or prevent contraction (FIGS. 8C-8E). In an expanded model, junction potential conductance was increased such that contractions were not restricted to slow wave peaks. Excitatory circular motor neurons innervated local circular muscle fibers with EJPs, and inhibitory circular motor neurons innervated local circular muscle fibers with IJPs. The model did not include longitudinal muscle fibers, because, unlike circular muscle, they do not directly drive pellet propagation, although longitudinal muscle fibers are known to be affected by peristaltic reflex pathways.

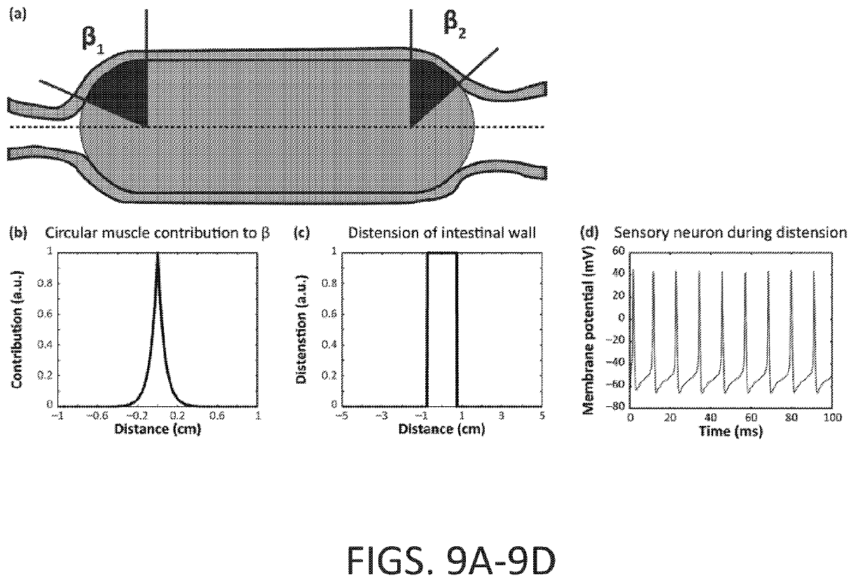

Virtual Pellet. Previous work modelled motility anatomically in three dimensions, although such anatomical models often lacked the physiological behavior to model electrical stimulation (see, e.g., Randhawa et al., 1996, Parsons and Huizinga, 2015, Du et al., 2016, Cheng et al., 2013). In this work, a virtual pellet was modeled as a 2D rigid object with position determined by fundamental mechanics. The pellet was a rectangle with semicircular endcaps and interacted with the intestinal wall through circular muscle tension. The top and bottom of the pellet were always in contact with the intestinal wall. The intestinal wall contacted each endcap at angle β₁ and β₂ (oral and anal endcap, respectively) as a function of circular muscle contraction (FIG. 9A). The extent of contact between the wall and pellet, observed as angle β, was the weighted sum of the tension in each muscle fiber. The weighted contribution of each muscle fiber to the contraction at either endpoint was a function of the distance between the muscle fiber and the endcap (FIG. 9B).

The interaction between the intestinal wall and the pellet at the endcaps (of radius R) applied a pressure (P) on the pellet. The pressure generated a force with an x-component that was a function of angle β(β₁ and β₂ for the oral and anal endcap, respectively) (equation 5(a)). The force on the pellet (F_{applied}) was the difference between the force acting on each of the endcaps (equation 5(b)). In addition to the applied force, there were static and kinetic friction forces (F_{static} & F_{kinetic}) that contributed to the net force (Fₙₑₜ) acting on the pellet, and these were functions of the static and kinetic coefficients of friction (F_{SØ} & F_{KØ}) (equation 5(c-e)). The net force on the pellet was used to determine the acceleration of the pellet and thus the velocity and position of the pellet, assuming no initial velocity and a known initial position.

F₁=P×R×(1−cos β₁) Equation 5(a)

F_{applied}∝ cos β₂−cos β₁ Equation 5(b)

F_{static}=F_{SØ} Equation 5(c)

F_{kinetic}=F_{KØ}×velocity Equation 5(d)

Fₙₑₜ=F_{applied}−(F_{static}+F_{kinetic} Equation 5(e)

The pellet interacted with sensory neurons through distension and an additional “stretch” current present in sensory neurons. Sensory neurons respond to stretching of the intestinal wall, as well as mucosal distortion. This model focused on stretch response, and it was modeled as an alpha synapse gated by stretch or distension. Distension was modeled as a binary variable at each point along the model: 1 at the position of the pellet, 0 otherwise (FIG. 9C). Later, in the expanded model, distension was modeled as a graded variable and sensory neurons responded proportionally to the magnitude of stretch. In other words, the stretch receptors detected the position of the pellet (FIG. 9D).

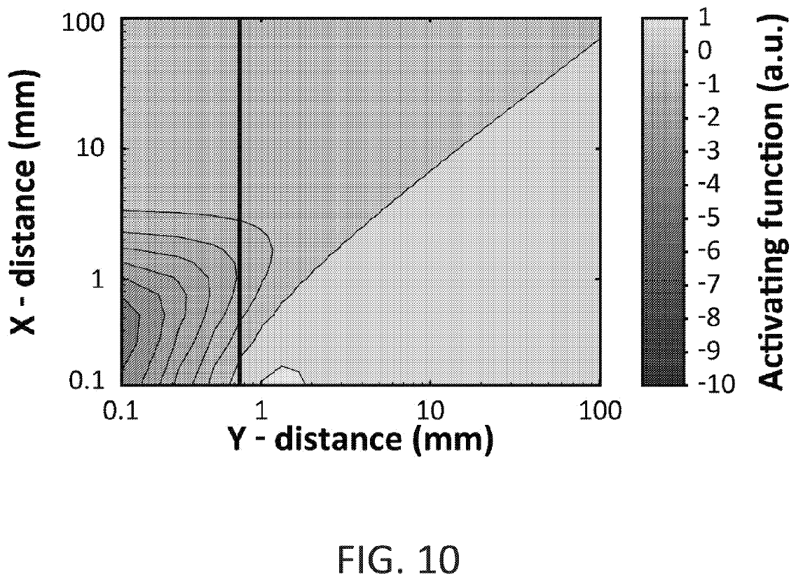

Electrical Stimulation. Electrical stimulation was simulated via a point electrode that applied an extracellular current to stimulate enteric neurons, smooth muscle fibers, and ICCs. The electrode stimulated cells by influencing the extracellular potential (Vₑ) at each cell as a function of electrode current (I_{electrode}), tissue conductivity (σ), and electrode-to-cell distance (equation 6(a)). Here, extracellular stimulation of point cells was modeled using cable theory with an activating function, assuming that all cells projected along the axial direction (x-direction). The effective stimulation current applied to each cell (Iₛₜᵢₘ) was proportional to the second derivative of extracellular potential with respect to the axial direction (x-direction) (equation 6(b)). The effective stimulation current was added to the derivative of the membrane potential with respect to time as an additional current term (equation 6(c)). A negative stimulation current created an activating function that depolarized or hyperpolarized cells depending on their position along the length of the simulated intestinal tract (x-direction) and distance between the tract and the electrode (y-direction) (FIG. 10).

$$\begin{matrix}{V}_{e} = \frac{{I}_{electrode}}{4 π σ \sqrt{{x}^{2} + {y}^{2}}} & Equation 6 ( a ) \\ {I}_{stim} = \frac{1}{{R}_{i}} \frac{{d}^{2} {V}_{e}}{{dx}^{2}} & Equation 6 ( b ) \\ \frac{{dV}_{m}}{dt} = \frac{{I}_{i o n} + {I}_{s y n} + {I}_{s t i m}}{{C}_{m}} & Equation 6 ( c )\end{matrix}$$

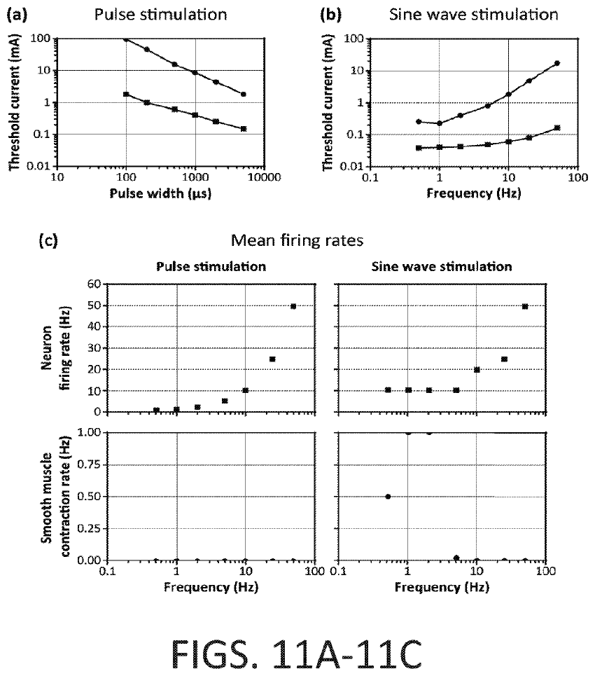

Approximations for extracellular electrical stimulation were validate by reproducing the strength-duration curve (see, e.g., Geddes and Bourland, 1985, Bostock et al., 1983). Stimulation current was applied to single cells to determine the minimum current necessary to evoke an action potential or contraction in enteric neurons and smooth muscle fibers, respectively, and threshold current decreased as pulse width increased (FIGS. 11A-11B). In enteric neurons, firing rate increased with stimulation frequency (FIG. 11C).

In vivo experimentation. The conclusions from the computational model were assessed experimentally by measuring colon transit time in awake rats. All animal care and procedures were approved by the Duke Institutional Animal Care and Use Committee. Female F344 rats (Charles River, 403), weighing between 150 and 200 grams, were selected at random for surgical and experimental procedures.

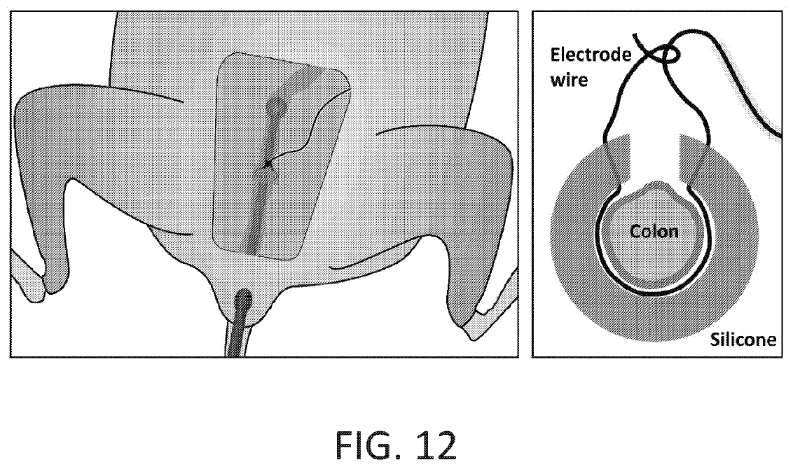

Surgical Procedure. Rats underwent antiseptic surgery to implant a stimulating electrode and a counter electrode. Rats were anesthetized with isoflurane: 3-4% v/v in oxygen to induce anesthesia and 1-2% to maintain anesthesia. The abdominal fur was shaved and the skin was cleaned with three alternating washes of iodine and alcohol. A sterile, ball-tipped probe was inserted intrarectally, 3 cm into the colon. Then, the abdominal cavity was opened, a cardiac pacing electrode (Medtronic, 6494) was threaded through a silicone collar, and the device was inserted beneath the descending colon. The active electrode wire was loosely wrapped around the colon, with enough slack to allow distension, and tied (FIG. 12). The muscle wall was closed with sutures, and a counter electrode was inserted into the subdermal space. Both wires were tunneled beneath the skin to the back of the neck, and they were tied to prevent the connector leads from retracting below the skin. The abdominal dermal incision was closed with sutures.

Colon Transit Time. A straight ball-tipped probe was inserted intrarectally 3 cm deep into the colon. The probe was withdrawn, and a 6-mm glass bead was inserted into the anus. The probe was then used to push the glass bead 3 cm deep into the colon. The time until the bead was expelled was measured under control and stimulated conditions. Both stimulation trials and control (no stimulation) trials were conducted in the same rat after a brief interval of approximately 2-5 minutes to reduce variability between individual rats and times of day. The order of the stimulation/control trial was randomized to account for changes in motility over repeated trials. Trials were repeated on four rats, with 15-20 minutes between pairs of trials. The experimental group was compared to the control group using a two-tailed paired t-test. Comparison between different stimulation groups was performed by comparing the percent change between each experimental trial and control trial across all experimental groups using an ANOVA and Tukey HSD post hoc tests. Significance level of α=0.05 was used for all statistical tests.

### 5. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Effects of Electrical Stimulation on Gut Motility

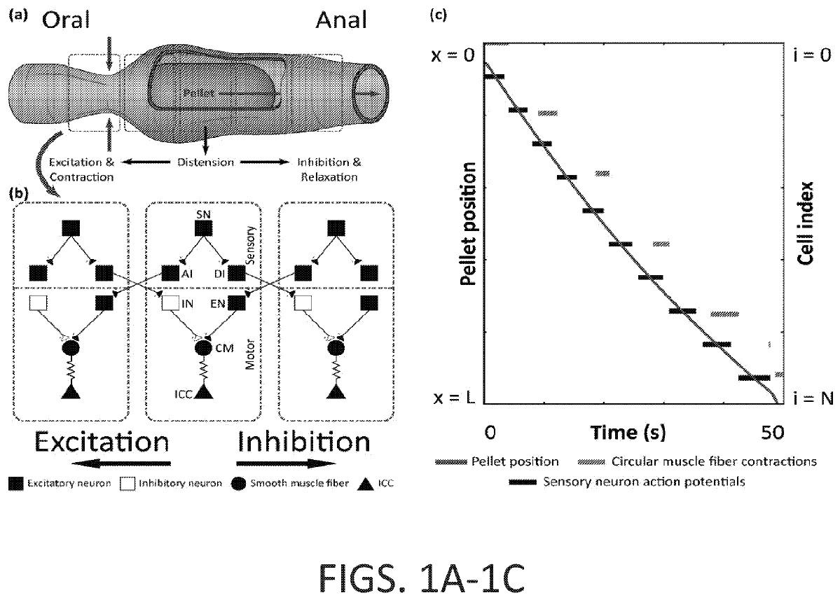

Embodiments of the present disclosure include an integrated neuromechanical model to determine the effects of electrical stimulation on intestinal motility. A virtual pellet was moved by smooth muscle contraction to simulate peristaltic propagation through a length of simulated intestinal tract, and sensory signals generated by interactions between the pellet and the gut wall were fed back to the network through sensory neurons (FIG. 1).

The pellet started with zero velocity at the oral end of the section, and the network activity, driven by the pacemaking cells (e.g., ICCs), caused the virtual pellet to propagate through the simulated tract. As shown in FIG. 1C, the position of the pellet is shown as a function of time. After 50 s of simulation, the pellet moved from the oral end to the anal end (x=L). The right vertical axis shows cell index, which corresponds to cell location (e.g., cell i=N is located at position x=L). Sensory neurons along the tract fired action potentials when the pellet reached their position and activated the stretch current by distension. The ascending excitatory pathway caused circular muscle fibers to contract oral to the stimulated sensory neurons.

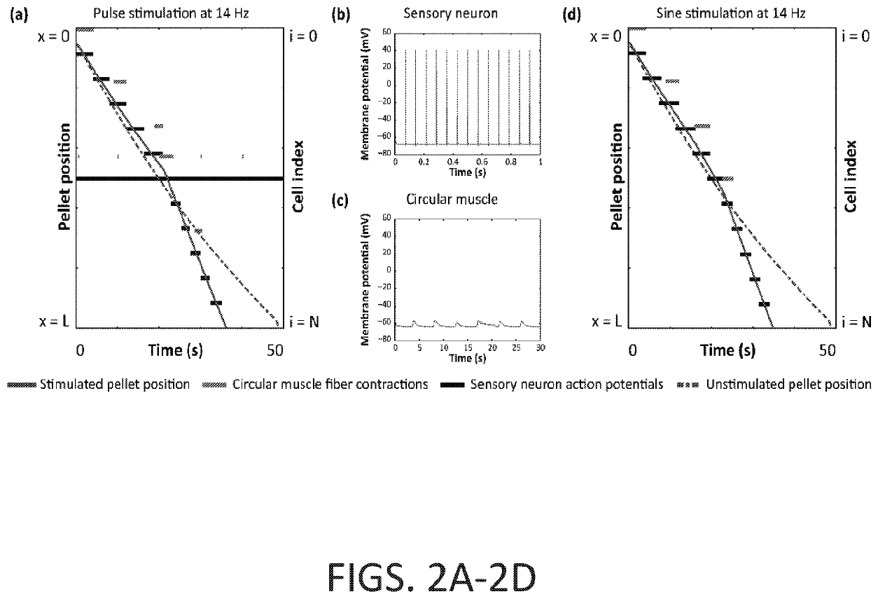

A simulated electrode was positioned on the simulated GI tract at a vertical offset (y) of 750 μm to determine the effects of electrical stimulation during peristalsis. The conclusions were not sensitive to the exact location of the electrode, and simulations with the simulated electrode positioned at the center of the simulated tract are shown (x=L/2). Electrical stimulation was applied continuously during each simulation. First, a 200 μs duration was tested with 1 mA current pulses repeating at 14 Hz, which is commonly used for neuromodulation (e.g., sacral nerve stimulation). Motility was measured by observing the time required to pass the virtual pellet through the simulated length. When current pulse stimulation was applied, the virtual pellet reached the anal end faster than without simulation (FIG. 2A). As shown in plots of threshold current of single cells (FIG. 11A), 1 mA 200 μs pulses were above the threshold current to generate neuron action potentials, but below the threshold for muscle fiber contractions. Consistent with the single-cell models, the applied stimulation evoked action potentials in enteric neurons (FIG. 2B), but did not directly cause muscle contraction (FIG. 2C).

Next, sine wave stimulation was tested, which is generally less efficient at activating neurons and rarely used for neuromodulation therapies (see, e.g., Swartz, C. (Ed.). (2009). Electroconvulsive and Neuromodulation Therapies. Cambridge: Cambridge University Press). Sine wave stimulation was applied at the same frequency and amplitude as pulse stimulation, 14 Hz and 1 mA. As expected, 14 Hz sine wave stimulation did not evoke action potentials in enteric neurons or cause muscle contractions. However, sine stimulation did decrease the transit time, compared to unstimulated peristalsis (FIG. 2D).

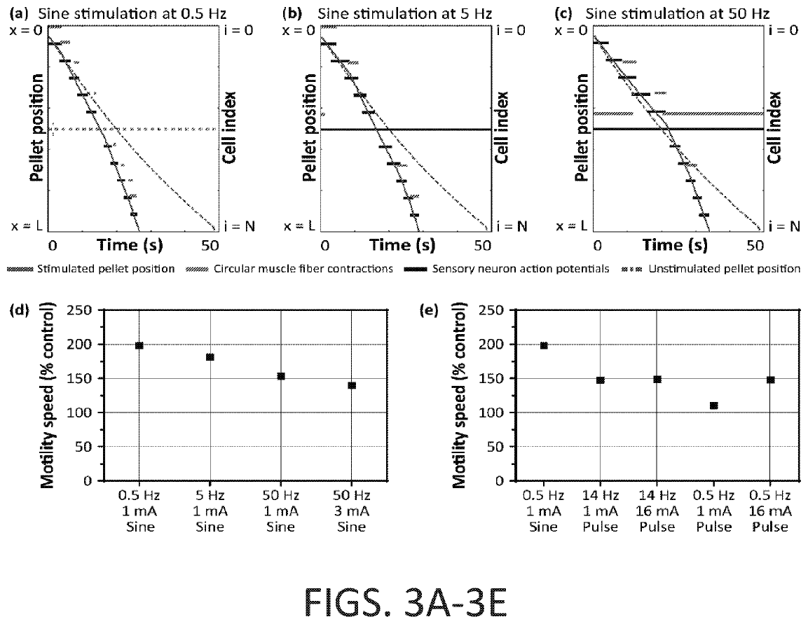

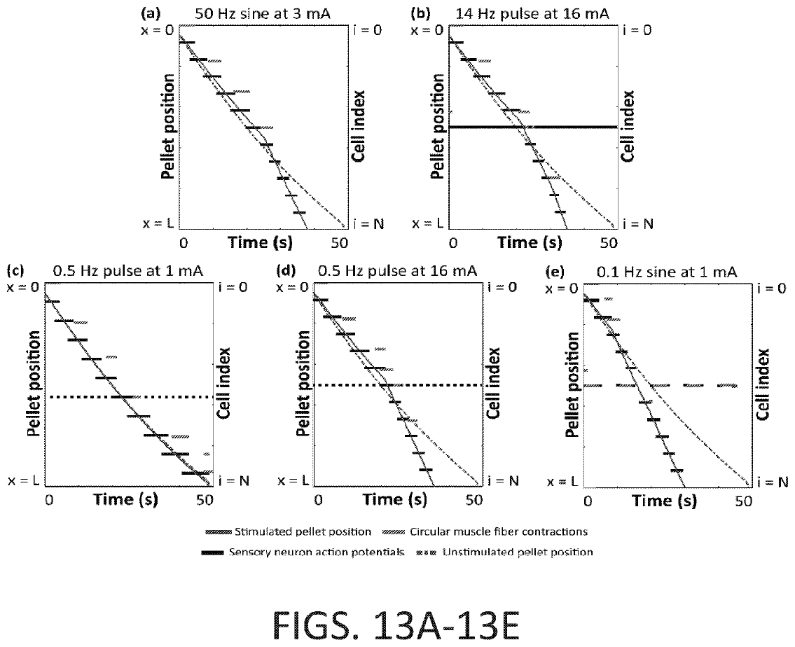

To understand further the effects of sine wave stimulation, a range of sine wave frequencies were tested: 0.5, 5, and 50 Hz at 1 mA (FIGS. 3A-3C). Each of these frequencies increased motility speed in the model. To account for different current thresholds at 50 Hz compared to 0.5 Hz and 5 Hz, stimulation at 50 Hz was repeated with amplitude scaled by ratio of the threshold currents (FIG. 13A). As shown, 0.5 Hz at 1 mA was an effective sine wave pattern for increasing motility speed, even when scaling amplitude for threshold current (FIG. 3D).

A 0.5 Hz sine wave stimulation was compared to 200 μs pulse stimulation applied at 14 Hz and 0.5 Hz, and at 1 mA and 16.3 mA, to account for the different threshold currents (FIGS. 13B-13D). Surprisingly, 0.5 Hz sine wave stimulation consistently increased motility speed compared to pulse stimulations at both frequencies and amplitudes (FIG. 3E), suggesting that electrical stimulation will be an effective means for entraining ICCs.

Results of the present disclosure demonstrate that the application of electrical stimulation increased gut motility as compared to non-stimulation, independent of the precise pattern of electrical stimulation applied. Additionally, results of the present disclosure demonstrate that an electrical pattern comprising a 0.5 Hz sine wave stimulation increased gut motility more than pulse stimulation; however, both sine and pulse stimulation increased gut motility as compared to no stimulation. This finding could not be explained by the effect of electrical stimulation on neurons or muscles alone (FIG. 11). Therefore, the effects of sine wave stimulation on the intrinsic pacemaker frequency of ICCs was examined further.

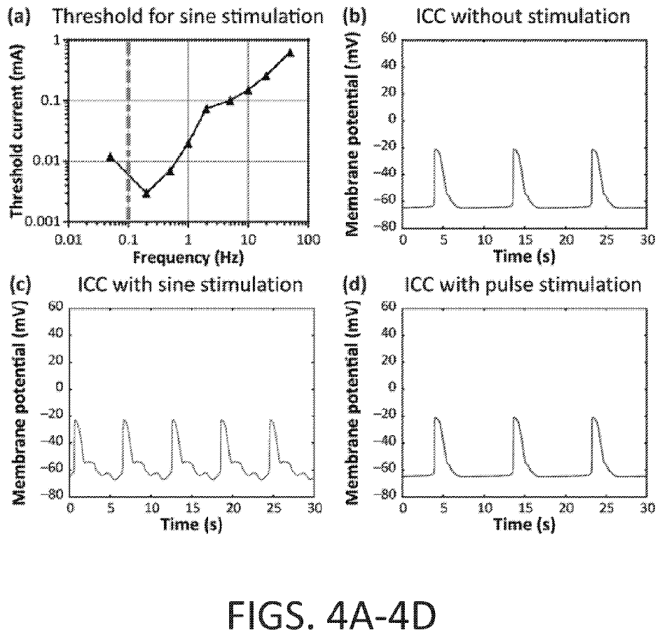

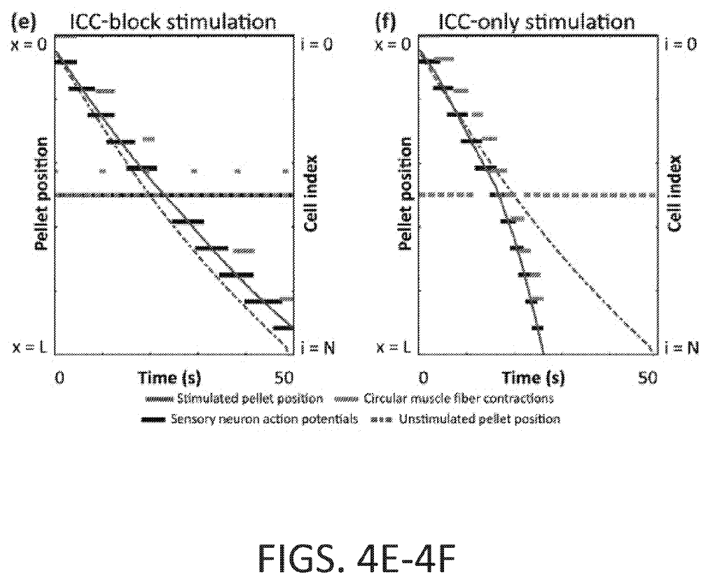

Sine wave stimulation was applied to ICCs over a range of frequencies from 0.05-50 Hz. As the current amplitude increased, the pacemaker frequency of ICCs was entrained to match the stimulation frequency. The threshold current for entraining the pacemaker frequency was determined over the range of sine wave frequencies (FIG. 4A), and the threshold current for entraining ICCs was lower at frequencies closer to 0.1 Hz, the natural pacemaker frequency of ICCs (FIG. 4B). Although low frequencies had a lower threshold for entrainment, they elicited a slower rate of contractions. Entraining ICCs to higher frequencies generated more contractions per second, thus increasing motility speed. According to modeling, 0.1 Hz sine wave stimulation entrained ICCs more easily, but 0.1 Hz sine wave stimulation increased motility speed less than 5 Hz sine wave stimulation, which had a markedly higher threshold for entrainment (FIG. 13E). Sine wave stimulation at 0.5 Hz was an effective because it had a low entrainment threshold while eliciting a high rate of contractions. The same ICC cell in the gut motility model was used to compare sine wave vs. pulse stimulation conditions. The ICC at position L/4 was modulated by 1 mA, 0.5 Hz sine wave stimulation (FIG. 4C), but unaffected by 1 mA, 200 μs pulse stimulation (FIG. 4D). This suggests that low-frequency sine waves can modulate the pacemaker frequency of ICCs over longer distances than pulse stimulation, and offers one explanation as to why 0.5 Hz sine wave stimulation was more effective at increasing motility speed than pulse stimulation.

To test further the role of ICCs in electrically stimulated peristalsis in the model, the extracellular stimulation term was removed from ICCs (equation 6(b)), so that electrical stimulation did not directly affect the ICCs but still affected other cells (e.g., enteric neurons and smooth muscle fibers). Removing ICC modulation abrogated the effect of 1 mA, 0.5 Hz sine wave stimulation on motility, as the pellet took longer to pass through the section than it did without stimulation (FIG. 4E). Stimulation was then restored to the ICCs, but removed from the model of enteric neuron and smooth muscle cells, so that only ICCs (but not other cells) were directly affected by electrical stimulation. In this model of ICC-only stimulation, the 1 mA, 0.5 Hz sine wave current was applied, and the pellet passed through the simulated section faster than without stimulation (FIG. 4F). This demonstrated that the ICCs played a critical role in the increase in gut motility by electrical stimulation.

The sensitivity of these results to key parameters in the model were evaluated, as shown in Table 1 below.

TABLE 1
Sensitivity analysis. Transit time for the unstimulated
model, as well as 0.5 Hz sine wave and 14 Hz pulse
stimulated model, with parameter adjustment.

| | Unstim- | 0.5 Hz | 14 Hz |
|---|---|---|---|
| | ulated | sine wave | 200 μs pulse |
| Parameter | transit | transit | transit |
| adjustment | time (s) | time (s) | time (s) |
| - Unadjusted - | 49.0065 | 24.5925 | 33.2251 |
| Electrode x-position: ↑ 50% | 49.0065 | 25.5126 | 51.4036 |
| Electrode x-position: ↓ 50% | 49.0065 | 18.1824 | 45.4658 |
| Electrode y-position: ↑ 50% | 49.0065 | 24.592 | 26.4177 |
| Electrode y-position: ↓ 50% | 49.0065 | 24.5923 | 45.3285 |
| Gap junction conductivity: ↑ 50% | 48.9868 | 22.5197 | 33.18 |
| Gap junction conductivity: ↓ 50% | 48.9893 | 24.3904 | 33.2852 |

Increasing or decreasing ICC gap junction conductance by 50% did not qualitatively change the effects of low frequency (0.5 Hz) sine wave stimulation or 14 Hz pulse stimulation on motility. Next, the x- and y-position of the electrode was adjusted by ±50%, and it was found that the relative effects of low frequency sine wave stimulation and 14 Hz pulse stimulation on transit time were qualitatively conserved.

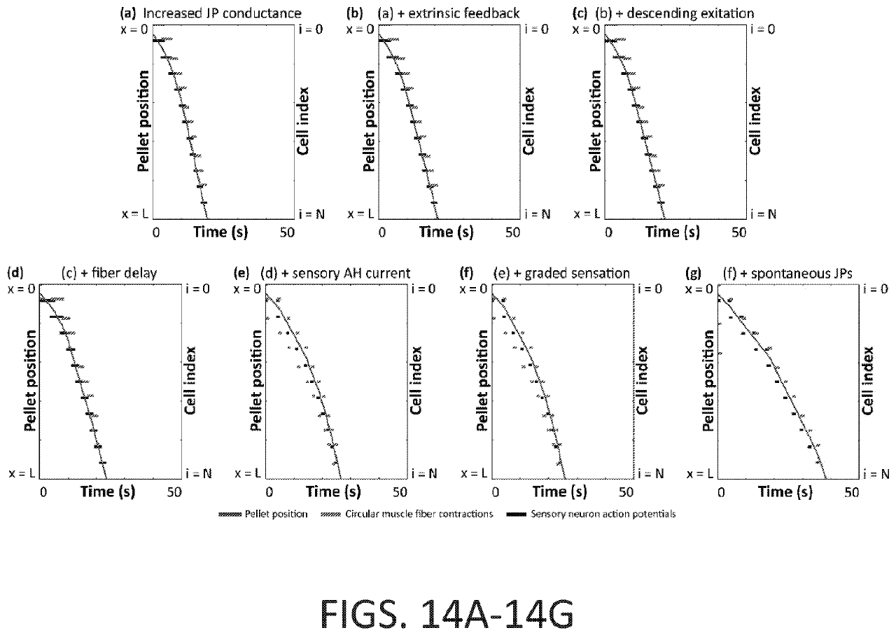

The robustness of these results was further tested by expanding the computational model to include additional neural and muscular mechanisms. First, IJP and EJP magnitudes in smooth muscle were increased, thus decreasing the influence of ICC slow waves on muscle contraction. In the unstimulated condition, this resulted in a decrease in transit time (FIG. 14A). Second, additional neural pathways for ascending inhibition and descending excitation were included. Ascending inhibition was activated by an extrinsic feedback loop, and descending excitation was initiated by intrinsic neurons. These two pathways alone did not markedly alter transit time in the unstimulated condition (FIGS. 14B-14C), but when fiber conduction delays were added based on conduction velocities previously described, the combined mechanisms slowed transit time (FIG. 14D).

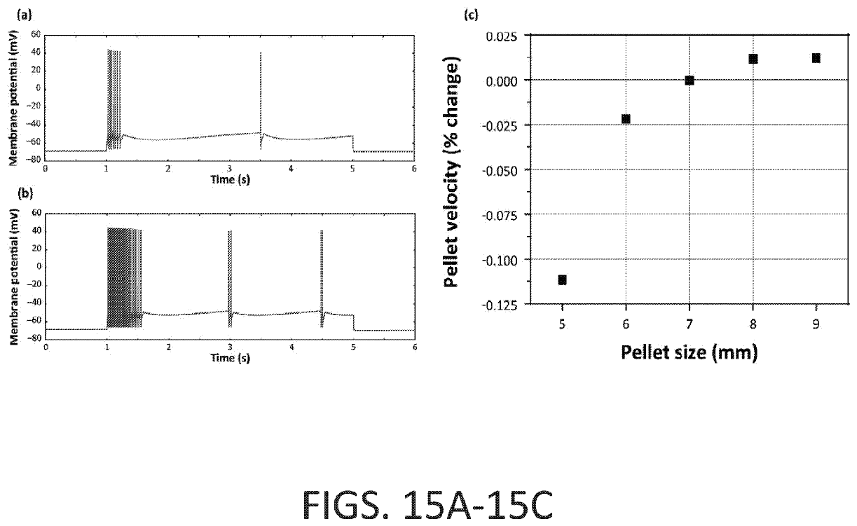

This model of intrinsic sensory neurons was then expanded to include the characteristic AH current. The AH mechanism limited the maximum firing rate of these cells and caused them to fire in short bursts, consistent with electrophysiology traces previously described and an established computational model (FIG. 15A). This dramatically increased transit time (FIG. 14E). A graded stretch response was also included in sensory neurons to reflect an increase in firing rate for larger stretch stimulations (FIG. 14F and FIGS. 15A-15B), which captured the relationship between pellet velocity and pellet size (FIG. 15C). Additionally, stochastic Poisson events were added for excitatory and inhibitory junction potentials based on circular muscle recordings previously described (FIG. 14G).

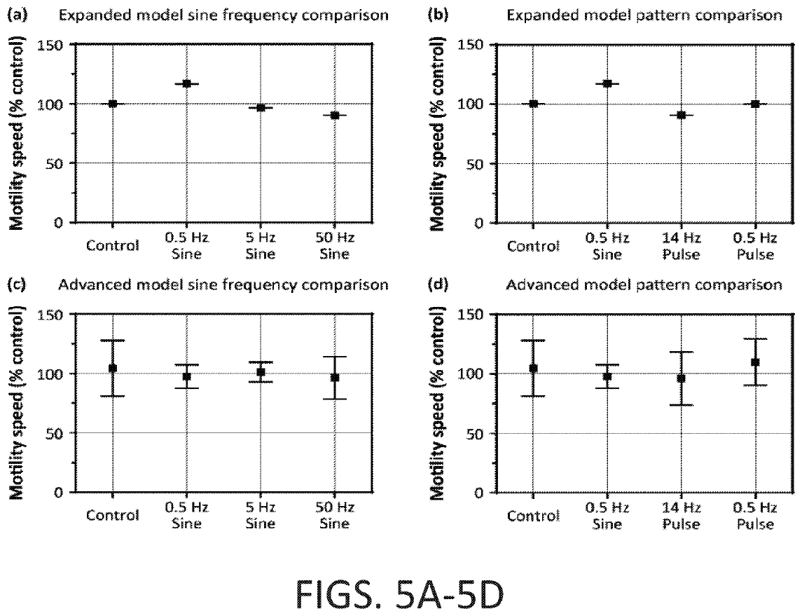

In this expanded model, spontaneous excitatory and inhibitory junction potentials introduced stochasticity. Simulation trials were repeated nine times for each stimulation group, and 0.5 Hz sine wave stimulation still increased motility speed more than 5 Hz and 50 Hz sine wave stimulation (FIG. 5A), and was more effective at increasing motility than 14 Hz and 0.5 Hz pulse stimulation (FIG. 5B). In the expanded model, the quantitative effects of electrical stimulation were somewhat diminished, which likely resulted from the additional inhibitory and motility-slowing mechanisms. However, 0.5 Hz sine wave stimulation remained an effective stimulation pattern among those tested, and qualitative trends were conserved in the expanded model. Only after increasing the synaptic weights of the added pathways, spontaneous firing rates, etc., by an order of magnitude were the effects of stimulation almost completely mitigated—under this condition, neither sine wave stimulation nor pulse stimulation had significant effects on motility speed (FIGS. 5C-5D). Therefore, the effectiveness of external stimulation depended on the relative strengths of the additional mechanisms and pathways, although 0.5 Hz sine wave remained an effective stimulation scheme.

### Example 2

### In Vivo Effects of Electrical Stimulation on Gut Motility

The computational models of the present disclosure indicated that electrical stimulation of the ENS increased gut motility and that these effects were somewhat dependent on the characteristic of the stimulus (e.g., pattern of electrical stimulation and corresponding frequency). This prediction was tested by measuring transit time of a glass bead inserted intrarectally into the colon of awake rats surgically implanted with a stimulation electrode. The time for the bead to travel 3 cm and exit the rectum was measured as a surrogate for colon transit time.

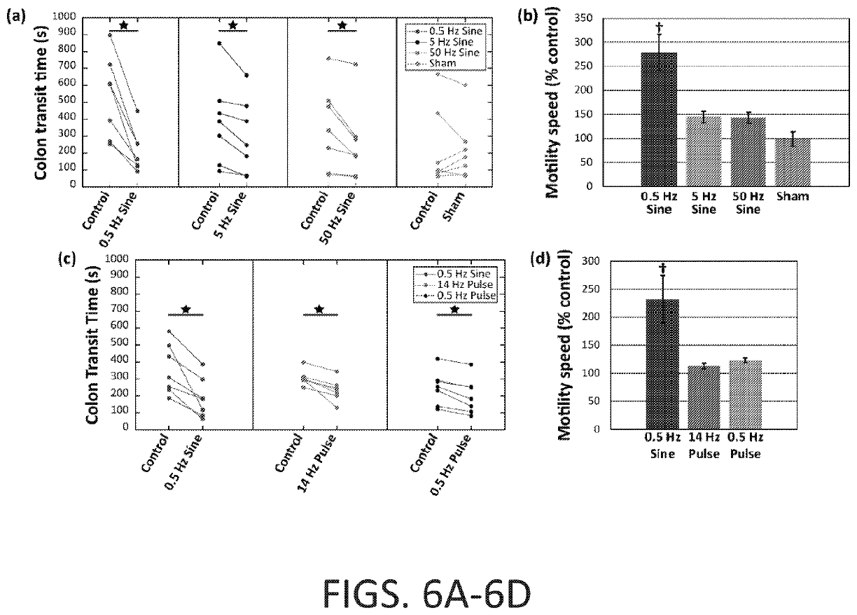

Colon transit time was reduced during sine wave stimulation as compared to colon transit time without stimulation. Four different stimulation groups were each compared to control: 0.5 Hz, 5 Hz, and 50 Hz sine wave stimulation at 1 mA, as well as a sham stimulation group (FIG. 6A). Transit time was significantly shorter in each stimulation group, 0.5 Hz, 5 Hz, and 50 Hz, compared to no stimulation. The sham stimulation group was not detectably different from control. When sine wave stimulation was compared between groups, 0.5 Hz increased motility speed more than all other groups (FIG. 6B).

Next, the effects of sine wave stimulation (0.5 Hz) were compared to the effects of pulse stimulation at 0.5 Hz and 14 Hz. In paired trials, all stimulation types reduced transit time compared to no stimulation (FIG. 6C), and 0.5 Hz sine stimulation increased motility speed more than pulse stimulation, consistent with model prediction (FIG. 6D). These results are consistent with the experiments performed with the computational modeling described above, and demonstrate that, in vivo, electrical stimulation is an effective means for entraining ICCs to treat gut dysmotility.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

What is claimed is:

1. A method of modulating gut motility in a subject, the method comprising applying a pattern of electrical stimulation to a portion of the subject's gastrointestinal tract, wherein the pattern of electrical stimulation entrains interstitial cells of Cajal (ICCs) in the portion of the subject's gastrointestinal tract and stimulates gut motility.

2. The method of claim 1, wherein the portion of the subject's gastrointestinal tract comprises the distal colon, rectum, or small intestine.

3. The method of claim 1, wherein the pattern of electrical stimulation comprises sinusoidal currents.

4. The method of claim 3, wherein the sinusoidal currents are applied at from about 0.1 Hz to about 5.0 Hz.

5-6. (canceled)

7. The method of claim 3, wherein the sinusoidal currents are applied at from about 0.1 mA to about 25.0 mA.

8. (canceled)

9. The method of claim 1, wherein the pattern of electrical stimulation comprises pulse stimulations.

10. The method of claim 9, wherein the pulse stimulations are applied at from about 0.1 Hz to about 20.0 Hz.

11. (canceled)

12. The method of claim 9, wherein the pulse stimulations are applied at about 14.0 Hz.

13. The method of claim 9, wherein the pulse stimulations are from about 100 μs to about 500 μs in duration.

14. (canceled)

15. The method of claim 9, wherein the pulse stimulations are about 200 μs in duration.

16. The method of claim 9, wherein the pulse stimulations are applied at from about 0.1 mA to about 25.0 mA.

17. (canceled)

18. The method of claim 1, wherein the method further comprises treating one or more symptoms of a functional gastrointestinal and/or motility disorder in the subject.

19. The method of claim 18, wherein the functional gastrointestinal and/or motility disorder is selected from the group consisting of: an esophageal disorder, a gastroduodenal disorder, a bowel disorder, centrally mediated disorders of gastrointestinal pain, gallbladder and sphincter of Oddi disorders, anorectal disorders, intestinal pseudo-obstructions, and childhood functional GI disorders.

20. (canceled)

21. The method of claim 1, wherein the one or more symptoms comprise gut dysmotility, and wherein the application of the pattern of electrical stimulation to the portion of the subject's gastrointestinal tract treats the dysmotility.

22. (canceled)

23. A method of treating a functional gastrointestinal disorder in a subject, the method comprising:
applying a pattern of electrical stimulation to a portion of the subject's gastrointestinal tract, wherein the pattern of electrical stimulation entrains interstitial cells of Cajal (ICCs) in the portion of the subject's gastrointestinal tract; and
treating one or more symptoms of a functional gastrointestinal and/or motility disorder in the subject.

24-35. (canceled)

36. The method of claim 23, wherein the one or more symptoms comprise dysmotility and the pattern of electrical stimulation comprises sinusoidal currents, and wherein the application of the electrical stimulation to the portion of the subject's gastrointestinal tract stimulates gut motility.

37. (canceled)

38. A method of operating an implantable neuromodulation device to treat a functional gastrointestinal and/or motility disorder in a subject, the method comprising:
configuring the neuromodulation device to apply a pattern of electrical stimulation to a portion of a subject's gastrointestinal tract, wherein the pattern of electrical stimulation entrains interstitial cells of Cajal (ICCs) in the portion of the subject's gastrointestinal tract; and
treating one or more symptoms of the functional gastrointestinal and/or motility disorder in the subject.

39. The method of claim 38, wherein the pattern of electrical stimulation comprises sinusoidal currents.

40. The method of claim 39, wherein the sinusoidal currents are applied at from about 0.1 Hz to about 5.0 Hz.

41. (canceled)

42. The method of claim 39, wherein the sinusoidal currents are applied at from about 0.1 mA to about 25.0 mA.

43. The method of claim 38, wherein the pattern of electrical stimulation comprises pulse stimulations.

44. The method of claim 43, wherein the pulse stimulations are applied at from about 0.5 Hz to about 15.0 Hz.

45. (canceled)

46. The method of claim 43, wherein the pulse stimulations are from about 100 μs to about 250 μs in duration.

47. (canceled)

48. The method of claim 43, wherein the pulse stimulations are applied at from about 0.1 mA to about 25.0 mA.

49. The method of claim 38, wherein the functional gastrointestinal and/or motility disorder is selected from the group consisting of: an esophageal disorder, a gastroduodenal disorder, a bowel disorder, centrally mediated disorders of gastrointestinal pain, gallbladder and sphincter of Oddi disorders, anorectal disorders, intestinal pseudo-obstructions, and childhood functional GI disorders.

50. (canceled)

51. The method of claim 38, wherein the one or more symptoms comprise dysmotility and the pattern of electrical stimulation comprises sinusoidal currents, and wherein the application of the electrical stimulation to the portion of the subject's gastrointestinal tract stimulates gut motility.

52. (canceled)
